(19) United States
(12) Patent Application Publication
(10) Pub. No.: US 2004/0134501 A1
(43) Pub. Date: Jul. 15, 2004

(54) Universal leg brace

(76) Inventors: Veronica Rose Hargis, Warwick, NY (US); Victor Khabie, Cross River, NY (US); William D. Thorpe, Newburgh, NY (US); Luther Calvin Hargis, Warwick, NY (US)

Correspondence Address: DR. LUTHER CALVIN HARGIS, 40 MAPLE AVE., WARWICK, NY, 10990, US

(21) Appl. No.: 10/724,860

(22) Filed: Dec. 1, 2003

Related U.S. Application Data
(60) Provisional application No. 60/433,093, filed on Dec. 16, 2002.

(51) Int. Cl. A61F 5/37

(52) U.S. Cl. 128/882

(57) ABSTRACT

An adjustable elastomeric leg support for knee, calf or thigh formed or a central body, interdigitating straps and terminal end tabs, secured by means of Velcro hook fasteners adjustably attachable to its elastomeric fabric exterior having a rubberized inner neoprene surface affording comfortable skin adherence that offers the capacity of fitting a variety of sizes and either leg while possessed of a unique leg brace design of interdigitating straps and opening passages to quickly and securely position, loosen or to readjust the brace while affording the capacity to simultaneously tension and bias the brace support system and patella with the use of inner buttresses if so desired.

### CROSS REFERENCES TO RELATED APPLICATIONS

This application claims the benefit of U.S. Disclosure Document 511239 and Provisional Patent 60/433,093.

### FIELD OF THE INVENTION

The present invention relates a method of bracing the leg with a non-conventional strapping device brace using interdigitating bands to support the leg, knee and patella with the ease and speed of simultaneous tensioning or re-adjustment of bilateral end tabs.

### BACKGROUND OF THE INVENTION

Man has attempted to stabilize and support injured or stressed body parts for centuries. The twentieth century has seen the evolution and advancement of many forms of tubular sleeve braces for the knee and leg as well as various forms of wrap around braces.

While many are suitable for their intended purpose they pose certain difficulties in their use and application that centers around the difficulty or exertion required to pull and place the tubular support about the intended body part as that brace must be snug to offer appropriate support and thus is often hard to pull into place. This is especially burdensome to the ill, handicapped or geriatric patient.

A further difficulty is the complexity of many braces with multiple straps and involved closure systems that are perhaps beyond the mechanical scope of many patients.

Another impediment is the difficulty in properly securing braces that are wrap around and whose underlying portion must be held by one hand while the overlapping second portion or side is secured. The clumsiness and often lack of proper security of tensioning is a common failing of the design.

Another disadvantage of most braces is their narrow range of fir requiring multiple brace sizes.

A final short coming is the ease with which many of these braces slip or shift shortly after placement of with tensioning and re-adjustment of complicated straps due to generally poor design or deficient skin adherence by the inner brace contact surface.

Accordingly what is needed is a brace that ‘goes on loose’ and is easily positioned, fits a variety of sizes, fits the left or right knee or leg, is non-complex and easy to use and reposition, can be re-adjusted quickly, can be easily tensioned by both hands simultaneously and lastly is comfortable and yet has good tissue compliance to avoid internal slippage.

### DESCRIPTION OF THE PRIOR ART

This invention relates generally to knee braces and more particularly to knee braces for use by the elderly.

Various knee or leg braces in the form of elastic sleeves are commercially available and/or are disclosed in the patent literature to stabilize or support the knee joint, patella and leg. Examples of such braces are disclosed in U.S. Pat. No. 3,703,171 (Schiavitto), U.S. Pat. No. 3,934,583 (Hollingshead), U.S. Pat. No. 3,945,046 (Stromgren), U.S. Pat. No. 3,970,081 (Applegate), U.S. Pat. No. 4,064,874 (Valin), U.S. Pat. No. 4,084,584 (Detty), U.S. Pat. No. 4,116,236 (Albert), U.S. Pat. No. 4,250,578 (Barlow), U.S. Pat. No. 4,296,744 (Palumbo), U.S. Pat. No. 4,353,362 (DeMarco), U.S. Pat. No. 4,366,813 (Nelson), U.S. Pat. No. 4,370,978 (Palumbo), U.S. Pat. No. 4,474,573 (Detty), U.S. Pat. No. 4,476,857 (Levine), U.S. Pat. No. 4,765,318 (Per Tranberg et al.), U.S. Pat. No. 5,085,210 (Smith), U.S. Pat. No. 5,139,477 (Peters), U.S. Pat. No. 5,168,577 (Detty), and U.S. Pat. No. 5,334,135 (Grim). While such sleeves are generally suitable for their intended purposes, a large segment of the population, namely, the elderly, may be incapable of adequately using such braces since they no longer have sufficient hand strength and/or dexterity to pull the sleeve onto their knee.

Various wrap-around knee braces are commercially available and/or disclosed in the patent literature. Examples of such braces are disclosed in U.S. Pat. No. 3,463,147 (Stubbs), U.S. Pat. No. 3,831,467 (Moore), U.S. Pat. No. 4,090,508 (Gaylord, Jr.), U.S. Pat. No. 4,651,722 (Karczewski), U.S. Pat. No. 4,378,009 (Rowley et al.), U.S. Pat. No. 5,024,216 (Hiono), U.S. Pat. No. 5,086,761 (Ingram), U.S. Pat. No. 5,221,252 (Caprio et al.), U.S. Pat. No. 5,399,153 (Caprio et al.), U.S. Pat. No. 5,451,201 (Prengler), and U.S. Pat. No. 5,472,413 (Detty).

### OBJECTS OF THE INVENTION

Accordingly, it is a general object of this invention to provide a leg brace which overcomes the disadvantages of the prior art.

It is a further object of this invention to provide a brace which is easy to apply and remove due to its interdigitating strap design and bilateral tab positioning.

It is a further object of this invention to provide a brace which is comfortable to wear and does not shift or slip on skin tissue.

It is a further object of this invention to provide a brace which provides good support for the knee structures.

It is a further object of this invention to provide a brace which serves to stabilize the knee joint and the patella.

It is a further object of this invention to provide a brace which when mounted is resistant to slippage, e.g., “riding-up” or sliding down.

It is a further object of this invention to provide a brace which does not bunch up in the back of the knee.

It is a further object of this invention to provide a brace, which when mounted in place acts like a sleeve brace.

It is a further object of this invention to provide a brace that will versatile and support the thigh, calf, knee or other body part.

It is a further object of this invention to provide a brace that will fit either leg.

It is a further object of this invention to provide a brace that will fit a variety of sizes due to its ability to expand or reduce in girth.

It is a further object of this invention to provide a brace that will allow for simultaneous and rapid adjustment or re-adjustment.

It is still a further object of this invention to provide a brace that allows for ease in biasing the patella.

### SUMMERY OF THE INVENTION

These and other objects of this invention are achieved by providing a leg brace for a person. The knee brace is formed of a generally planar sheet of elastomeric, e.g., fabric coated neoprene material having a central body-portion, with or without a patellar opening depending on its use as a knee, thigh or calf brace (or other body part). The elastomeric sheet comprises a central portion giving rise to three straps on one side and two on the other which pass through suitable openings to become unified as lateral or end tabs after interdigitating. The tab ends of the brace includes a free end portion in the form of a releasable securable connector.

The central portion of the brace is arranged to be disposed on the person's knee so that the longitudinal axis is disposed over the person's patella if used as a knee support. A patellar opening fits the circumference of the patellar in registration with same and a surrounding inner Velcro velour doughnut allows for the positioning of two inner patellar semi-lunar support buttress if so desired.

Due to the adjustability of size afforded by the interdigitating strap design the brace can be quickly slipped over the foot in its unsecured and expanded position and tightened rapidly and securely at any position along the leg or other body part.

### BRIEF DESCRIPTION OF THE DRAWINGS

FIG. 1 is a frontal and slightly downward view of the support prior to closure.

FIG. 2 is a frontal view of the support in its closed position.

FIG. 3a is a cross sectional view of the brace on the leg in its closed position.

FIG. 3b is an enlarged cross sectional view of the supports neoprene elastomeric fabric.

FIG. 4 is an inner view of the brace's central area to depict its patellar opening and buttresses.

FIG. 5 is a rearward view of the support to depict its position of smallest size adjustment.

FIG. 6 is a rearward view of the support to depict its position of maximum size adjustment.

FIG. 7 is a frontal view of the brace being positioned for knee support.

FIG. 8 is a frontal view of the brace being positioned for thigh support and rotated in this case to clarify the rearward band components of the brace that pass through or ‘interdigitate’ one another.

FIG. 9 is a frontal view of the brace being positioned for calf support without patellar opening.

FIG. 10 is a frontal view of a brace variation.

FIG. 11 is a frontal view of the design of FIG. 10 in the closed position for knee support.

FIG. 12 is a rearward view of the brace of FIG. 10 depicting the passage of lateral brace components.

### DETAILED DESCRIPTION OF THE PREFERRED EMBODIMENTS

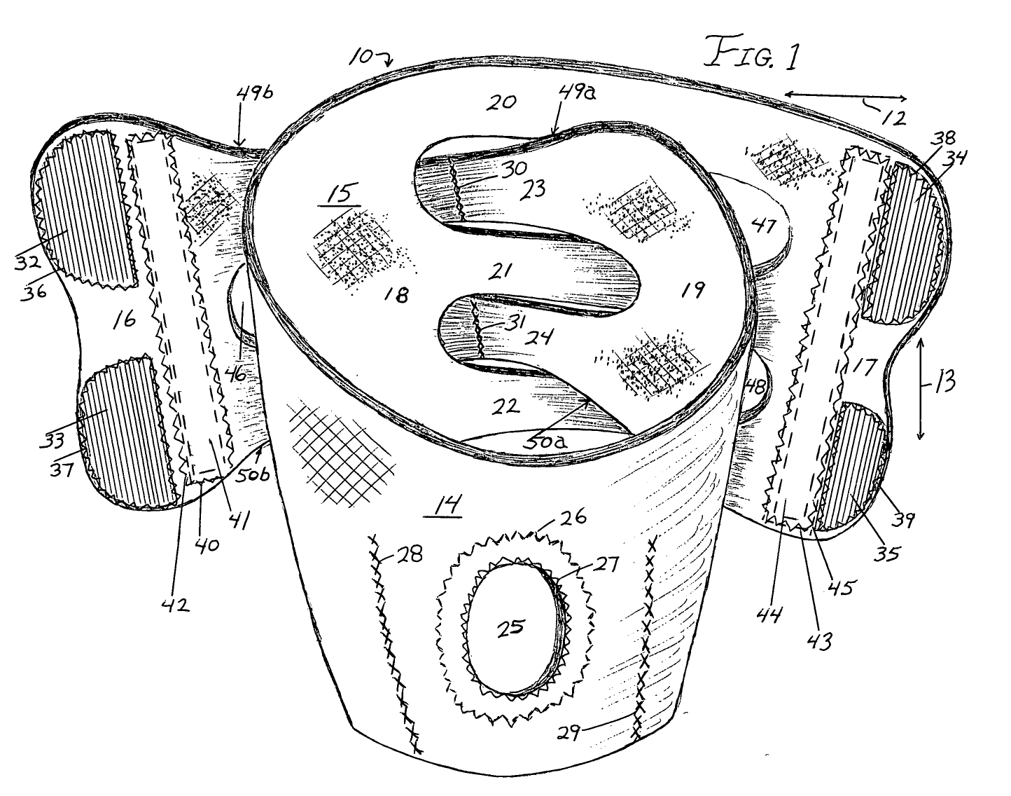

FIG. 1 shows a knee support 10. The support composed primarily of plush neoprene (fabric outer layer bonded to a neoprene inner layer). The support is shown according to the present invention in its unsecured position having a given height 13 and length 12. A central outer portion 14 of the support is positioned over the kneecap or patellae 25. An inner portion 15 having two distinct sides 18 and 19 that give rise to rearward bands 20, 21, 22 and 23, 24 respectively. These bands passing through corresponding openings in the rearward part of the brace 46, 47 and 48. A sloping or narrowing of the band 23 and 24 is depicted by 49a and 50a allowing their unimpeded movement through opening 47 and 48. Areas 49b and 50b indicate a flaring of the straps 23 and 24 that allows for some expansion to allow the end tab 16 more contact surface for closure of the Velcro bell hook fabric parts 32 and 33. These being stitched to the neoprene at 36 and 37. On the opposite end or tab 17 having Velcro bell hook closure fabric 34 and 35 stitched by 38, 39. Each end or tab being reinforced by a flexible stay indicated by 41 and 44 and housed by their respective cloth covers 40 and 43. These covers stitched by 42 and 45. Straps 23 and 24 are joined to their more terminal ends by the abutment of their pieces at a conjoined juncture and stitching indicated by 30 and 31 to allow attachment of tab end 16 to side 19.

The front portion of the support depicts the patellar opening 25 as previously described. The brace is gathered slightly and diminished to better contour the infra-patellar leg region at joined and stitched points 28 and 29. A velour fabric or multi-loop VELCRO.RTM area around the inner patellar opening on the inner aspect of the brace is attached by stitching at its inner and outer circumference depicted by 27 and 26.

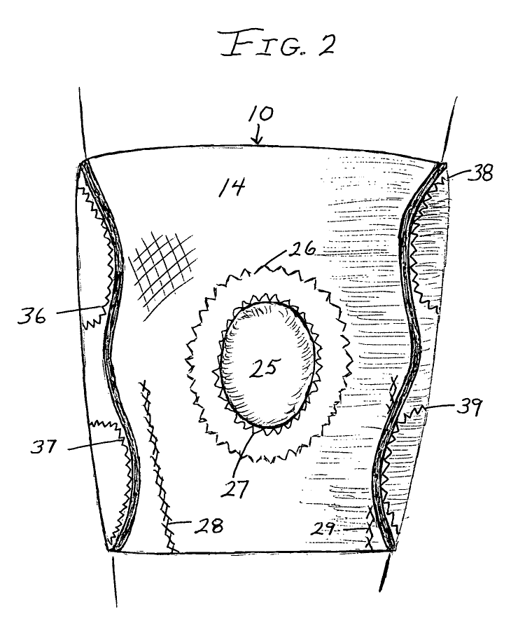

FIG. 2 references the support 10 secured about the knee. Area 25 in registration with the patellae, with stitching 26, 27 as a means of securing the inner plush buttress fabric, area 28 and 29 as a means of joining the lower brace for proper knee contour and 36, 37, 38 and 39 as stitching for Velcro closure tabs.

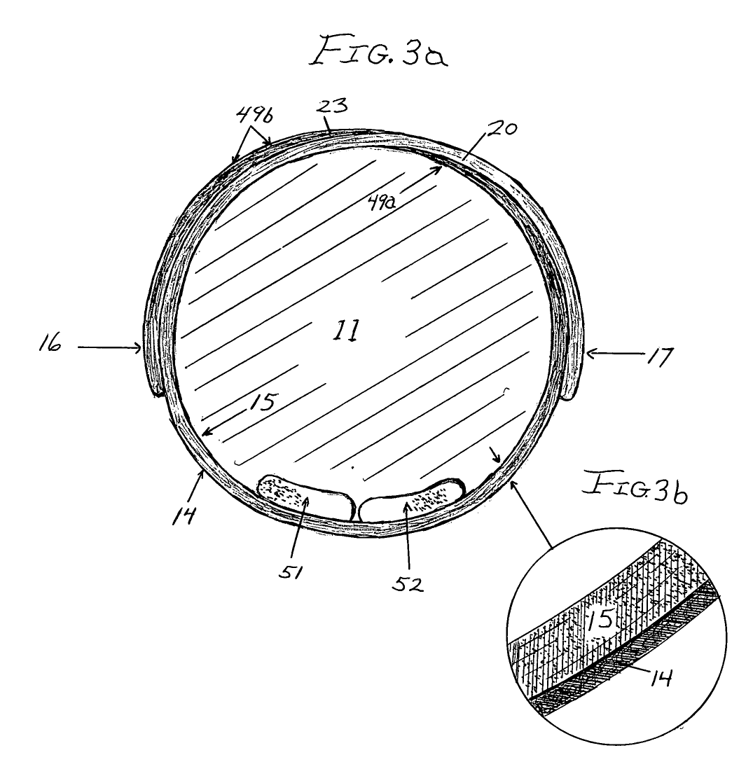

FIG. 3a where-in the cross sectioned leg is seen as 11 with the front brace 14 representing the outer brace plush fabric surface of bonded neoprene. The brace of area 14 extending backward to become 20 and 23 strap components, ending in terminal edges or tabs 17 and 16. 49a and 49b represent sloping contours of strap 23. Items 51 and 52 represent portions of the internal buttresses. Area 15 denoting the inner aspect of the brace which is the inner surface of the bonded neoprene being the rubberized surface.

FIG. 3b representing and enlarged cross section of the bonded neoprene elastomeric brace material to depict the inner surface 15 which is the rubberized neoprene proper and area 14 which is the elastomeric plush fabric covering of lesser width used to allow a suitable surface for attachment of the bell hook Velcro closures.

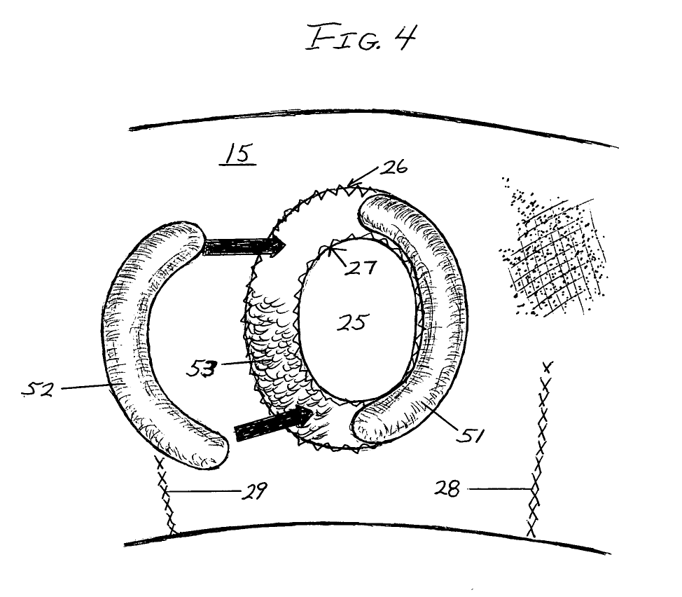

FIG. 4 to view the inner aspect of the support 10, which reveals rearward elastomeric rubberized neoprene 15, velour plush or multi-loop VELCRO.RTM closure fabric doughnut 53 surrounded and adhered to 15 by stitching 26 and 27, patellar opening 25, contouring stitches 28 and 29, and semi-lunar buttresses 51 and 52.

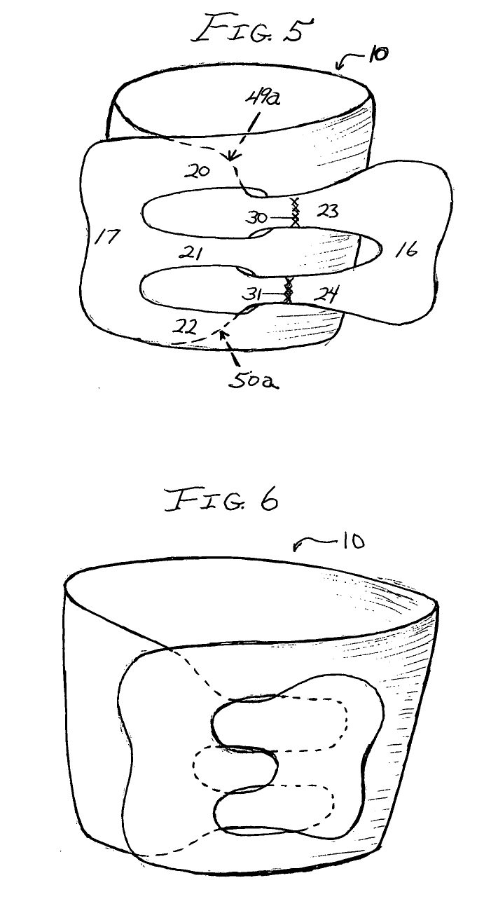

FIG. 5 reveals the brace 10 in its smallest size to demonstrate straps 23 and 24 of part 16 and straps 20, 21 and 22 of part 17. Joining and stitches of strap 23 and 24 are shown as 30 and 31 respectively. Rearward and hidden areas of contour emerging from straps 23 and 24 beneath straps 20 and 22 are shown as areas 49a and 50a.

FIG. 6 discloses the support 10 in its largest dimension.

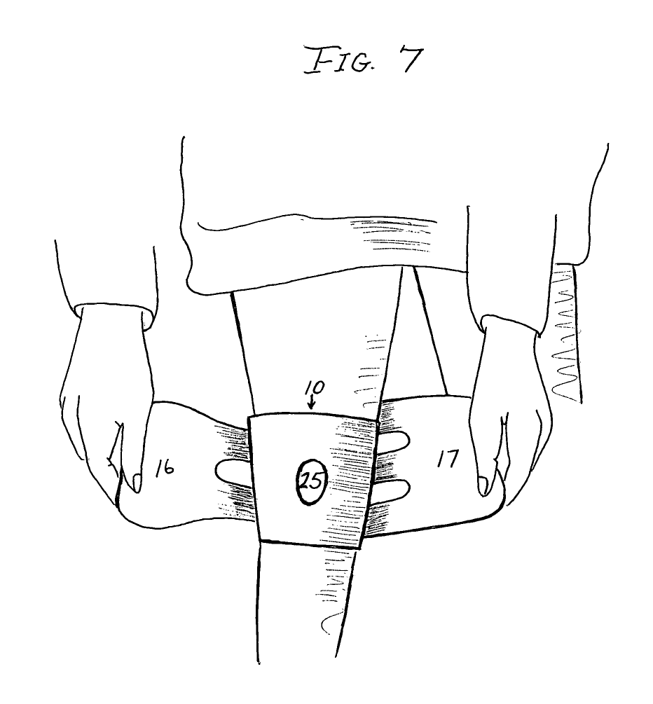

FIG. 7 is a frontal view of the brace placed to support the knee with area 25 in registration with the patellae. The brace is positioned but not yet secured and can be seen to be grasped by its ends/tabs as 16 and 17 respectively.

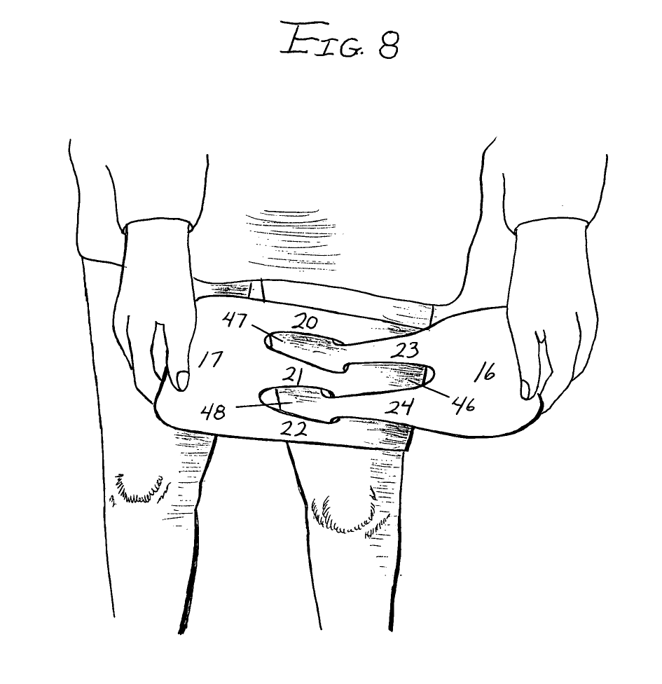

FIG. 8 is a rearward view of the brace rotated 180 degrees to reveal the backward strap interdigitation mechanism unique to this leg brace; particularly tab end 17 becoming straps 20, 21 and 22 separating openings 47 and 48 to allow passage of straps 23 and 24 evolving from tab end 16. It is understood that body portion 14 surrounds the rearward/unseen knee. Space 46 allows for passage of the middle strap 21 of tab 17.

FIG. 9 is a frontal view of the positioned but unsecured brace of 10 as used for calf support. In this instance as in support of the upper leg no patellar opening is needed and so would be deleted in brace manufacture. Seen are tab ends 16 and 17 with passage openings 46, 47 and 48.

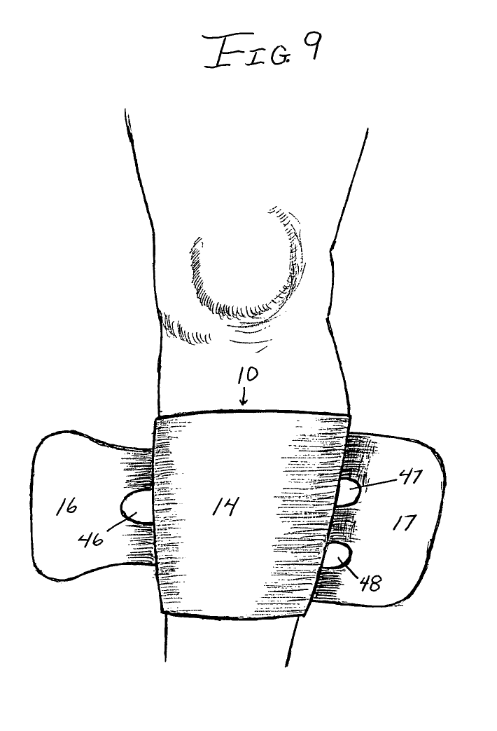

FIG. 10 is a frontal view of an alternate strap design which allows for passage of part 54 through opening 58. The space of 58 allows for upper and lower straps 56 and 57 leading to tab end 55 with stitching 59 for underlying Velcro. The patellar opening 25 is shown but may be deleted in cases of manufacture for thigh and calf use. Depicted is stitching 26 and 27 and body portion 14. The opposite tab end depicts stitching 60 for underlying Velcro.

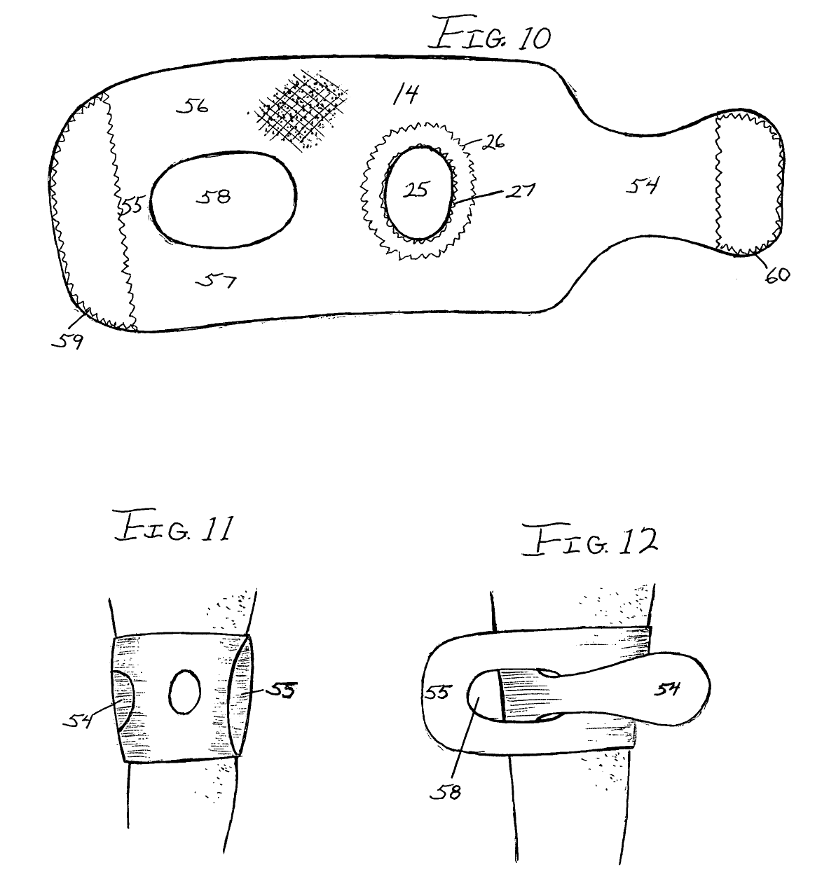

FIG. 11 is a frontal view of the design of FIG. 10 that depicts the brace positioned and secured for knee support. Secured tab ends 54 and 55 are seen.

FIG. 12 demonstrates the brace of FIG. 10 and shows the positioned but unsecured brace having the passage of 54 through space 58 and alternate end tab 55.

As a practical matter the positioning or readjustment of the brace can be quickly afforded by bilateral and simultaneous loosening of the lateral or terminal end tab contact surfaces of bell hook Velcro to the braces plush outer fabric. As will be appreciated from the foregoing, the removal of the knee brace 10 can be readily achieved by even persons having very little hand strength and/or dexterity/coordination. In particular, to accomplish that task, all that the user has to do is to grasp the end tabs 16/17 and peel them away from the plush fabric 14 on the surface of the brace to which they are releasable secured. This action releases each tab from connection, thereby freeing the associated end tab. When all the tabs are free, the brace can be taken off the knee or quickly re-adjusted.

It should be pointed out at this junction that other materials than those described heretofore can be used to make the brace of this invention. For example, elastomeric materials other than neoprene having a tufted fabric covering may be used. Thus, the neoprene may not be covered by any fabric or may be covered by a non-tufted fabric, such as smooth nylon. In the case where the material of the sheet of material forming the brace does not include a plush fabric covering, the outer surface of that sheet should include at least patches of a plush or multi-loop VELCRO.RTM Moreover, in some cases, it may not be desired to use neoprene as the material of the brace. Thus, other elastic materials, with or without cushioning and thermal retention properties may be used for either, if desired. Further still, the central portion 10 and tabs 16 and 17 need not be a unitary member, i.e., formed of a single piece of material. Thus, one or more of those portions of the brace may be formed of plural pieces which are secured together to form an integral assembly.

As should be appreciated from the foregoing, the brace of the subject invention can be applied to various sized limbs. This can be performed without requiring undue stretching or manipulation. This feature makes it particularly suitable for a variety of sizes and for geriatric applications. Moreover, the adjustment straps of the brace provide customized tensioning of the brace above and below the knee, while leaving the patella unconstrained by any strap stretched there over (as has characterized several prior art knee braces). Moreover, the opposite direction wrapping of the end tabs provides increased balance and stability for the brace. Further still, the application of the brace can be achieved quite easily and simply and does not require complicated wrappings of long straps. All that is required is to pull and stretch the various tabs and subsequently straps and secure the fastening means. The application of customized bilateral tension to the brace allows for an ease of application and a fit and feel of brace support quite unlike any other brace. Moreover, the construction of the knee brace of this invention is very simple. In this regard it can be readily fabricated from any suitable material(s), e.g., a sheet of fabric covered neoprene.

Without further elaboration, the foregoing will so fully illustrate my invention, that others may, by applying current or future knowledge; readily adapt the same for use under the various conditions of service.

What is claimed is:

1. A leg brace for a person, said leg brace being formed of a generally planar sheet of elastomeric material and having a pair of adjustment ends or tabs connected by interdigitating straps to a central body portion. The tab ends secured thereby, to said braces outer elastomeric fabric surface by means of Velcro fabric bell hook closures located at the inner surface of the tab ends.

2. The brace comprising a central portion and having a girth and height to support a calf, knee, thigh or other body part.

3. A brace whose interdigitating strap design allows for the rapid, secure deployment and fitting of the brace by simultaneously tensioning its two tab ends.

4. A brace that allows for the securing of same in registration with the patellae when used as a knee brace and biasing of the patella due to simultaneous tab tensioning.

5. A brace that allows for the support of the thigh or calf without patellar opening when used for same.

6. A brace who's adjustable girth allows it to fit of a wide range of sizes.

7. A brace whose design permits its use on either leg.
